(19) United States
(12) Patent Application Publication
(10) Pub. No.: US 2026/0102114 A1
(43) Pub. Date: Apr. 16, 2026

(54) Stimulating a User During Sleep Based on Sleep Stages Predicted From Sensor Data of a Wearable Device

(71) Applicants: Samsung Electronics Co., Ltd., Suwon-si, Gyeonggi-do (KR); Massachusetts Institute of Technology, Cambridge, MA (US)

(72) Inventors: Wei Ting Samantha Chan, Cambridge, MA (US); Craig Ferguson, Lexington, MA (US); Nelson Hidalgo Julia, Boston, MA (US); Viswam Nathan, Clovis, CA (US); Nnamdi Obi, Greenville, NC (US); Anita Podrug, New York, NY (US); Tanmay Srivastava, Selden, NY (US); Varun Kumar Viswanath, Fremont, CA (US); Nathan Whitmore, Cambridge, MA (US); Zhijian Yang, Nutley, NJ (US)

(21) Appl. No.: 19/359,112

(22) Filed: Oct. 15, 2025

Related U.S. Application Data
(60) Provisional application No. 63/707,694, filed on Oct. 15, 2024.

(51) Int. Cl. A61B 5/00

(52) U.S. Cl. CPC A61B 5/4812; A61B 5/681

(57) ABSTRACT

In one embodiment, a method includes obtaining, by one or more sensors of a wearable device worn by a user, one or more biological signals of the user and determining, based on the one or more biological signals, a current sleep stage of the user. The method further includes determining, based at least on the determined sleep stage of the user, whether to provide a stimulation to the user; and in response to a determination to provide a stimulation to the user, then providing, by one or more electronic devices in the environment of the user, the stimulation to the user.

### PRIORITY CLAIM

This application claims the benefit under 35 U.S. C. § 119 of U.S. Provisional Ser. No. 63/707,694 , filed Oct. 15, 2024, which is incorporated by reference herein.

### TECHNICAL FIELD

This application generally relates to stimulating a user during sleep based on sleep stages predicted from sensor data of a wearable device.

### BACKGROUND

In humans, sleep can be divided into several distinct stages. For instance, the following sleep stages may be used to define sleep in humans: (1) a wakeful, non-sleep stage; (2) an initial light-sleep stage, also referred to as an “N1” stage; (3) a subsequent stage of deeper light sleep, also referred to as an “N2” stage; (4) a slow-wave, deep-sleep stage, also referred to as an “N3” stage; and (5) a rapid-eye movement, or “REM,” sleep stage, although there are other ways of defining human sleep stages. A typical night of sleep in an adult human can include 4-6 rounds of sleep cycles, with each sleep cycle including the four stages 2-5 described above.

### BRIEF DESCRIPTION OF THE DRAWINGS

FIG. 1 illustrates an example of a process that uses a wearable device to sense a sleep stage of a user, and then based on the sensed sleep stage, provides the user with stimulation to improve the user's sleep.

FIG. 2 illustrates technique for skipping windows of sensor data and inferring the skipped data.

FIG. 3 illustrates an example computing system.

### DESCRIPTION OF EXAMPLE EMBODIMENTS

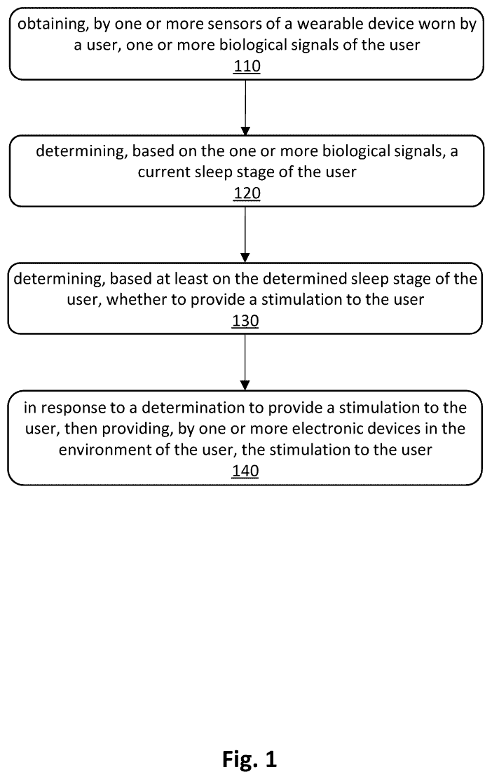

FIG. 1 illustrates an example of a process that uses a wearable device to sense a sleep stage of a user, and then based on the sensed sleep stage, the user can be provided with stimulation to improve the user's sleep. Step 110 of the example method of FIG. 1 includes obtaining, by one or more sensors of a wearable device worn by a user, one or more biological signals of the user. The wearable device may include a wrist-worn device (such as a smartwatch, bracelet, or wristband), a ring, a necklace, or any other suitable wearable device. The sensors may include for example, one or more of an accelerometer, a gyroscope, a light sensor, a barometer, a temperature sensor, a heart rate sensor, and/or a photoplethysmography (PPG) sensor. The one or more sensors of the wearable device obtain biological data, which may be stored in a memory of the wearable device and/or provided to another electronic device, such as a client device (e.g., a personal computer, a smartphone, etc.)

In particular embodiments, data from at least some sensors may be recorded at a specified interval (e.g., every 12 seconds). In particular embodiments, data from at least some sensors may be recorded whenever a sensed value changes (e.g., whenever an accelerometer records a change in acceleration). Sensor data can be converted to a constant sampling rate format by extrapolating the last known value of each sensor until the next update is registered. The data can then be resampled at a chosen frequency. The maximum achievable sampling rate is defined by the underlying hardware sampling rate and depends on the particular device used. A sensor may operate at the maximum sampling rate, or alternatively may operate at a lower sampling rate, e.g., to reduce power use.

Step 120 of the example method of FIG. 1 includes determining, based on the one or more biological signals, a current sleep stage of the user. In particular embodiments, step 120 may be performed by the wearable device itself. For example, a smartwatch may include sensors that detect biological signals of the wearer, and this sensor data may be logged and processed by the smartwatch. The smartwatch may include, e.g., a trained machine-learning model (as described below) for predicting a current sleep stage of the user. In other embodiments, all or some of the processes of step 120 may be performed by another computing device, such as a client device (e.g., a smartphone) or a server device that obtains the sensor data from the wearable device (e.g., over a wired or wireless connection, such as over a Wi-Fi connection, etc.) or from an intermediary electronic device.

In particular embodiments, step 120 may include classifying a current sleep stage of the user as one of a predetermined set of sleep-stage classification labels. For example, particular embodiments may use a set of five sleep-stages classification labels, e.g.,: wake (S0), S1, S2, S3, and REM. Stages S1, S2, and S3 may also be referred to as N1, N2, and N3, respectively. This classification may be performed by, for example, a trained machine-learning model that is trained on training sensor data and corresponding ground-truth sleep-stage labels, as explained below.

In particular embodiments, sleep-stage estimation may be based on one or more of data from a heart-rate sensor, an accelerometer, and a gyroscope. Each sensor may produce data about three axes, e.g., axes in Cartesian coordinates, although other coordinate systems may be used in addition or in the alternative. In particular embodiments, sensor data (e.g., accelerometer and gyroscope data) may be normalized to the maximum observed in the training data (as described below) so that the maximum value for sensor data (e.g., about each axis of that sensor's coordinate system) is 1.

In particular embodiments, a user's current sleep stage may be estimated based on a window of previous sensor data, such as a 30-second window of data, a 1-minute window of data, etc. A sleep stage may be estimated at particular intervals, e.g., every 1 minute, every 10 seconds, every 1 second, etc., and these intervals may be associated with particular sleep stages, in that the estimation interval can vary based on the sleep stage the user is estimated to currently be in, as explained more fully below.

In particular embodiments, time-domain sensor data may be transformed into the frequency domain using, e.g., Fast Fourier Transforms (FFTs). For example, an FFT may be calculated for the heart rate, and also for the accelerometer signal summed across all axes and the gyroscope signal summed across all axes. These FFTs are calculated over, e.g., 64-sample windows (approximately one minute of data) at a 1 Hz frequency, resulting in 64 features each for heart rate, accelerometer (sum of X, Y, and Z axes), and gyroscope data (sum of X, Y, and Z axes). In addition, the values of each accelerometer and gyro axis in the previous second may also be given as input, for a total of 199 features in this example. Since the FFTs are calculated once per second over a 64-sample window, the window is a sliding window over the 64 most recently acquired samples of the sensor data, in an example in which a 1-minute window of data is used.

Features derived from the sensor data are then input to a classifier, such as a trained neural network classifier. For example, a neural network classifier may contain four layers: three dense hidden layers with 128, 64, and 32 units respectively, each using rectified linear unit (ReLU) activation functions, followed by a dense output layer with 5 units and a softmax activation function, although this disclosure contemplates that other classifier architectures may be used.

In particular embodiments, the machine-learning model used to predict the current sleep stage of the user may be trained based on training data from one or more of a heart-rate sensor, an accelerometer, and/or a gyroscope. For example, data from each of those sensors may be acquired from test subjects while the test subject's ground-truth sleep stages are determined, e.g., using a polysomnography device. Features can then be extracted from the sensor data, and this training data and corresponding ground-truth classifications may then be used to train the machine-learning model used to predict the user's sleep stage during the real-time inference of step 120.

Step 130 of the example method of FIG. 1 includes determining, based at least on the determined sleep stage of the user, whether to provide a stimulation to the user. For example, as described below, if the user is determined to be in a deep-sleep (e.g., a stage S3 or N3 sleep-stage), then certain stimulation(s) may be applied to the user in order to enhance the user's sleep or sleep-related benefits.

In particular embodiments, the current sleep stage of the user as predicted by a trained classifier may be used to determine whether stimulation should be applied to the user. For example, if the user's sleep-stage is classified as a deep-sleep stage, then stimulation may be applied to the user, as described below. In other embodiments, sleep-stage classification may be a necessary but not sufficient condition to apply stimulation. For example, stimulation may only be applied when the user is determined to be in a deep-sleep stage, and in addition one or more sleep metrics of the user exceeds one or more corresponding thresholds. For example, stimulation may be applied when one or more of the following sleep metrics exceeds a corresponding threshold.

As one example, the probability of the user being in a deep-sleep stage (e.g., N3 stage) in the current second must be above a particular threshold, such as a user-specified or medical-professional-specified threshold, before stimulation will be applied. As another example, the average probability of deep-sleep stage over the past predetermined time period (e.g., a predetermined number of seconds) must be above a particular threshold, such as a user-specified or medical-professional-specified threshold. As another example, at least a predetermined time period (e.g., a predetermined number of seconds) must have passed since sleep onset, where sleep onset may be detected by the operating system of the wearable computing device.

In other example, at least a predetermined time period (e.g., a predetermined number of seconds) must have passed since the last detected arousal by the user. For instance, an arousal may be defined by one or more of the following: a large motion being detected by the wearable device; the probability of N3 sleep-stage in the current second dropping below a particular (e.g., user specified) threshold; or the average probability of being in an N3 sleep-stage over a predetermined time period (e.g., a predetermined user-specified number of seconds) dropping below a particular threshold. A large motion may be defined in any suitable way. For example, a large motion may mean that the sum of the normalized values of the three axes of the gyroscope exceeds 3 degrees per second, although other threshold values may be used.

In other example, no more than a predetermined number of sleep stimuli (i.e., the number of stimuli resulting from step 140) can be presented in order to present another stimulus, e.g., to avoid overstimulating the user while the user is asleep. In another example, the user must have been in a stable sleep state for at least a predetermined amount of time (e.g., a user-specified number of seconds). A stable sleep state may be defined in any suitable way. For example, a stable sleep state may be defined based sleep metrics including (1) the probability the user's current sleep state, (2) the probability of the user's sleep state over a particular period of time, (3) an amount of time since sleep onset, and (4) a number of arousals, where each of these (1)-(4) sleep metrics must meet its corresponding threshold for a particular period of time (e.g., over the past 60 seconds) for the user's sleep state to be considered stable.

In another example, no more than a predetermined amount of time (e.g., a user-specified number of seconds) can have elapsed since the start of sleep. The start of sleep may be detected in the same manner as described above for onset of sleep.

Particular embodiment may use one or more of the sleep metrics to determine whether to provide stimulus to a user at a particular moment, even when the user has meet the sleep-stage classification requirements of step 130. As described above, the sleep-stage metrics may include a number of variable thresholds, and therefore the stimulation process can be tuned to a particular user. For example, the decision of whether to apply stimulation can be tuned between sensitivity and specificity; e.g., for a person or population that has severe sleep issues, then the threshold(s) may prioritize stimulation so as to improve sleep quality, while for a person or population who has generally good sleep quality, then the threshold(s) may be tuned so as to prioritize not waking that person up (e.g., by stimulating only based on a very high probability of the user being in a stable deep sleep).

In particular embodiments, some or all of step 130 may be performed by the wearable device. In other embodiments, some or all of step 130 may be performed by another electronic device, such as a client device or a server device.

Step 140 of the example method of FIG. 1 includes in response to a determination to provide a stimulation to the user, then providing, by one or more electronic devices in the environment of the user, the stimulation to the user. The stimulation may be a sound or vibration, as described below, although this disclosure contemplates that any suitable stimulation may be applied. In particular embodiments, a stimulation may be applied by the wearable device (i.e., the wearable device may be one of the one or more electronic devices in the environment of the user). For example, a sound may be played by a speaker of the wearable device, or a vibration may be induced by the wearable device. In other embodiments, the stimulation may be applied by another device. For example, a sound may be played by a speaker (e.g., in a smartphone, a smartspeaker, etc.) in the same room as the user. In particular embodiment, a first wearable device (e.g., a smartwatch) may include the sensors referenced in step 110, while another wearable device performs at least part of step 140 (e.g., a ring may provide vibrations to a user).

Particular embodiments may perform step 140 by providing stimulation for slow wave entrainment. Here, the stimulation may be pulses of sound (e.g., white noise), where the pulse frequency has approximately the same frequency as brain waves that occur during deep sleep. This pulse frequency may be in the range of 0.5 Hz to 4 Hz in particular embodiments, although other pulse frequencies may be used. Particular embodiment may use a pulse frequency of 1.2 Hz.

In particular embodiments, step 140 may include applying vibrations (e.g., by the wearable device) to the user. Like the example above, the vibrations may be applied in pulses with a frequency in a range of 0.5 Hz-4 Hz, similar to the frequency of brain waves in deep sleep. By providing acoustic or vibrational stimulation to the user during deep sleep that approximates the frequency of brain waves during sleep, then the depth and quality of the user's sleep may be improved, which may reduce awakenings and arousals that disrupt sleep quality.

In particular embodiments, step 140 may include a targeted memory reactivation technique, in which a cue can be associated with an experience that occurs during wake, and then that cue can be presented as stimulation during stage 3 sleep. For example, a user who is studying for a test may choose to have a periodic vibration stimulation (e.g., from a wearable device) occur while studying. This vibration stimulation is the cue in the targeted memory reactivation technique. Then, when that same user experiences stage-3 sleep, that same vibration stimulation is applied to the user. The vibration may be, e.g., a relatively weak vibration that occurs every 10 seconds, although other stimulations, application periods, and stimulations intensities may be used. As a result of targeted memory reactivation, the user's memory and recall of the experience may be improved.

In particular embodiments, stimulation intensity may vary while step 140 is being performed. For example, stimulation intensity (e.g., the loudness of an acoustic sound or the vibrational energy of a stimulating vibration) may start at a low initial value and then gradually increase (e.g., linearly increase) during a stimulation episode. In particular embodiments, if an arousal is detected while stimulation is occurring, then the intensity of the stimulation may be capped at the value that triggered the arousal, reduced by a small offset. Stimulation can then continue at this capped intensity until either the end of the sleep epoch (where the cap is reset, in particular embodiments) or until another arousal occurs (at which point the cap and intensity may be decreased further). A sleep epoch refers to the entire period of sleep of the user, for example from going to sleep at night to waking up in the morning. In particular embodiments, a stimulation's initial value, the type of increase (e.g., linear, logarithmic, etc.), and the capped value may be set by a user, and in particular embodiments may be specific to the type of stimulation applied and/or to the device applying the stimulation.

In particular embodiments, one or more of steps 110-120 may be performed with a frequency that depends on the most-recently detected sleep stage of the user. For example, if the user is detected to be awake or in a light sleep stage, then step 120 may occur every, e.g., 5 minutes, while if the user is detected to be in an N2 or N3 sleep stage, then steps 120 may occur more frequently (e.g., every 10 seconds or every second), thereby reducing power consumption when the user is in sleep stages that are not suitable for applying stimuli to the user. Likewise, particular embodiments may vary the frequency of steps 110 and/or 120 based on the time of day and the user's typical sleeping habits. For example, deep sleep can be more likely to occur relatively earlier in a sleep epoch (e.g., in the first 2-3 hours), and so steps 110 and/or 120 may occur relatively more frequently during the early portion of a sleep period compared to the end of the sleep period. Either or both the sleep stage and the time of day may also be used to vary the application of a window-skipping technique, for example the window-skipping technique described below.

Particular embodiments may reduce power consumption of the example method of FIG. 1 by duty cycling the times during which sensor data is collected and processed. For example, a CPU typically consumes the most power in a smartwatch, and therefore the CPU may be turned off during times that the CPU would otherwise be required to perform processing functions related to sensor data acquisition and processing. Skipped windows of sensor data can then be inferred so that sleep-stage determines (e.g., step 120) can still be performed while the CPU is turned off.

Particular embodiments use a trained neural network to identify windows to skip by analyzing past sensor data. This approach takes advantage of the predictive capabilities of recurrent networks, such long short-term memory (LSTM) networks, to turn off the CPU. However, the predictive model needs to be run multiple times throughout the day and night to make predictions, adding to battery consumption.

A different approach divides the sensor data into segments, with each segment made up of, e.g., 10 one-minute windows. For each segment, the power management module skips a particular number of windows (e.g., three windows, thereby reducing the duty cycle to 70%).

After acquiring the sensor data (e.g., PPG data), then the skipped data can be inferred in order to estimate what the data would have been. Here, the inferred data may be raw sensor data or may be features derived from that sensor data, e.g., to provide to a trained ML model to determine a user's sleep stage as in step 120. For example, one estimation technique uses the nth segment's first 7 windows (in an example in which the last 3 windows are skipped) and the n+1th segment's first 7 windows to infer the nth segment's last 3 windows. While some embodiments may use polynomial interpolation to infer skipped data, other embodiments may take a more sophisticated approach.

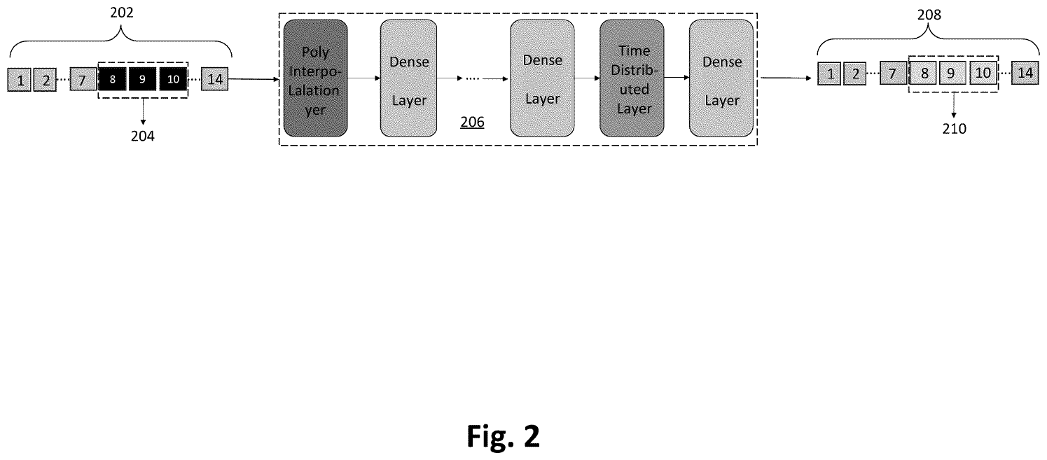

FIG. 2 illustrates one such technique for skipping windows of sensor data and inferring the skipped data. In the example of FIG. 2, a residual convolutional neural network (CNN) model 206 is trained to reduce the difference between interpolated data and true data. Sensor data 202 (e.g., sensor features) include skipped windows 204. Residual CNN 206 makes an initial estimate using polynomial interpolation. Residual CNN 206 is trained to refine these initial estimates, allowing the model to capture complex, non-linear patterns in the sensor features while benefiting from the polynomial interpolation's strengths. For example, CNN 206 may include a series of 1D convolutional layers that extract features and patterns from the entire sequence, including both the original and interpolated values. After flattening the convolutional output, dense layers process this information to predict residuals for the middle three values. The final prediction features 208, including estimated data 210, are obtained by adding these residuals to the initially interpolated values, thus refining the polynomial interpolation based on learned patterns in the data. As described above, in particular embodiments all the interpolation operations may be performed on sensor features instead of raw sensor signals, for instance in order to reduce errors in sleep-stage estimation.

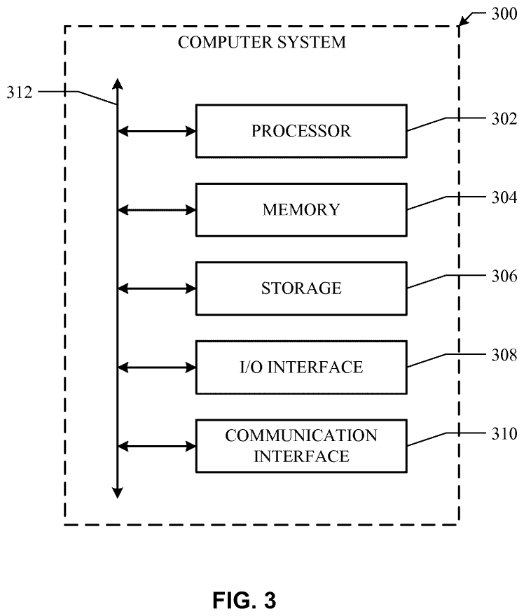

FIG. 3 illustrates an example computer system 300. In particular embodiments, one or more computer systems 300 perform one or more steps of one or more methods described or illustrated herein. In particular embodiments, one or more computer systems 300 provide functionality described or illustrated herein. In particular embodiments, software running on one or more computer systems 300 performs one or more steps of one or more methods described or illustrated herein or provides functionality described or illustrated herein. Particular embodiments include one or more portions of one or more computer systems 300. Herein, reference to a computer system may encompass a computing device, and vice versa, where appropriate. Moreover, reference to a computer system may encompass one or more computer systems, where appropriate.

This disclosure contemplates any suitable number of computer systems 300. This disclosure contemplates computer system 300 taking any suitable physical form. As example and not by way of limitation, computer system 300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 300 may include one or more computer systems 300; be unitary or distributed; span multiple locations; span multiple machines; span multiple data centers; or reside in a cloud, which may include one or more cloud components in one or more networks. Where appropriate, one or more computer systems 300 may perform without substantial spatial or temporal limitation one or more steps of one or more methods described or illustrated herein. As an example and not by way of limitation, one or more computer systems 300 may perform in real time or in batch mode one or more steps of one or more methods described or illustrated herein. One or more computer systems 300 may perform at different times or at different locations one or more steps of one or more methods described or illustrated herein, where appropriate.

In particular embodiments, computer system 300 includes a processor 302, memory 304, storage 306, an input/output (I/O) interface 308, a communication interface 310, and a bus 312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 302 includes hardware for executing instructions, such as those making up a computer program. As an example and not by way of limitation, to execute instructions, processor 302 may retrieve (or fetch) the instructions from an internal register, an internal cache, memory 304, or storage 306; decode and execute them; and then write one or more results to an internal register, an internal cache, memory 304, or storage 306. In particular embodiments, processor 302 may include one or more internal caches for data, instructions, or addresses. This disclosure contemplates processor 302 including any suitable number of any suitable internal caches, where appropriate. As an example and not by way of limitation, processor 302 may include one or more instruction caches, one or more data caches, and one or more translation lookaside buffers (TLBs). Instructions in the instruction caches may be copies of instructions in memory 304 or storage 306, and the instruction caches may speed up retrieval of those instructions by processor 302. Data in the data caches may be copies of data in memory 304 or storage 306 for instructions executing at processor 302 to operate on; the results of previous instructions executed at processor 302 for access by subsequent instructions executing at processor 302 or for writing to memory 304 or storage 306; or other suitable data. The data caches may speed up read or write operations by processor 302. The TLBs may speed up virtual-address translation for processor 302. In particular embodiments, processor 302 may include one or more internal registers for data, instructions, or addresses. This disclosure contemplates processor 302 including any suitable number of any suitable internal registers, where appropriate. Where appropriate, processor 302 may include one or more arithmetic logic units (ALUs); be a multi-core processor; or include one or more processors 302. Although this disclosure describes and illustrates a particular processor, this disclosure contemplates any suitable processor.

In particular embodiments, memory 304 includes main memory for storing instructions for processor 302 to execute or data for processor 302 to operate on. As an example and not by way of limitation, computer system 300 may load instructions from storage 306 or another source (such as, for example, another computer system 300) to memory 304. Processor 302 may then load the instructions from memory 304 to an internal register or internal cache. To execute the instructions, processor 302 may retrieve the instructions from the internal register or internal cache and decode them. During or after execution of the instructions, processor 302 may write one or more results (which may be intermediate or final results) to the internal register or internal cache. Processor 302 may then write one or more of those results to memory 304. In particular embodiments, processor 302 executes only instructions in one or more internal registers or internal caches or in memory 304 (as opposed to storage 306 or elsewhere) and operates only on data in one or more internal registers or internal caches or in memory 304 (as opposed to storage 306 or elsewhere). One or more memory buses (which may each include an address bus and a data bus) may couple processor 302 to memory 304. Bus 312 may include one or more memory buses, as described below. In particular embodiments, one or more memory management units (MMUs) reside between processor 302 and memory 304 and facilitate accesses to memory 304 requested by processor 302. In particular embodiments, memory 304 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 304 may include one or more memories 304, where appropriate. Although this disclosure describes and illustrates particular memory, this disclosure contemplates any suitable memory.

In particular embodiments, storage 306 includes mass storage for data or instructions. As an example and not by way of limitation, storage 306 may include a hard disk drive (HDD), a floppy disk drive, flash memory, an optical disc, a magneto-optical disc, magnetic tape, or a Universal Serial Bus (USB) drive or a combination of two or more of these. Storage 306 may include removable or non-removable (or fixed) media, where appropriate. Storage 306 may be internal or external to computer system 300, where appropriate. In particular embodiments, storage 306 is non-volatile, solid-state memory. In particular embodiments, storage 306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 306 taking any suitable physical form. Storage 306 may include one or more storage control units facilitating communication between processor 302 and storage 306, where appropriate. Where appropriate, storage 306 may include one or more storages 306. Although this disclosure describes and illustrates particular storage, this disclosure contemplates any suitable storage.

In particular embodiments, I/O interface 308 includes hardware, software, or both, providing one or more interfaces for communication between computer system 300 and one or more I/O devices. Computer system 300 may include one or more of these I/O devices, where appropriate. One or more of these I/O devices may enable communication between a person and computer system 300. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 308 for them. Where appropriate, I/O interface 308 may include one or more device or software drivers enabling processor 302 to drive one or more of these I/O devices. I/O interface 308 may include one or more I/O interfaces 308, where appropriate. Although this disclosure describes and illustrates a particular I/O interface, this disclosure contemplates any suitable I/O interface.

In particular embodiments, communication interface 310 includes hardware, software, or both providing one or more interfaces for communication (such as, for example, packet-based communication) between computer system 300 and one or more other computer systems 300 or one or more networks. As an example and not by way of limitation, communication interface 310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 310 for it. As an example and not by way of limitation, computer system 300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 300 may include any suitable communication interface 310 for any of these networks, where appropriate. Communication interface 310 may include one or more communication interfaces 310, where appropriate. Although this disclosure describes and illustrates a particular communication interface, this disclosure contemplates any suitable communication interface.

In particular embodiments, bus 312 includes hardware, software, or both coupling components of computer system 300 to each other. As an example and not by way of limitation, bus 312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 312 may include one or more buses 312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
obtaining, by one or more sensors of a wearable device worn by a user, one or more biological signals of the user;
determining, based on the one or more biological signals, a current sleep stage of the user;
determining, based at least on the determined sleep stage of the user, whether to provide a stimulation to the user; and
in response to a determination to provide a stimulation to the user, then providing, by one or more electronic devices in the environment of the user, the stimulation to the user.

2. The method of claim 1, wherein the wearable device comprises a wrist-worn device.

3. The method of claim 2, wherein the one or more sensors comprise one or more of a PPG sensor, a heartrate sensor, an accelerometer, or a gyroscope.

4. The method of claim 3, wherein the stimulation comprises one or more of a vibration or a sound.

5. The method of claim 4, wherein the one or more electronic devices comprise the wearable device.

6. The method of claim 1, further comprising:
determining, based on the one or more biological signal, that the user is currently in a deep-sleep sleep stage; and
in response to the determination, then providing an acoustic stimulation or a vibrational stimulation to the user.

7. The method of claim 6, wherein the acoustic stimulation comprises one or more acoustic pulses or one or more vibrational pulses having a pulse frequency in a range of 0.5 Hz to 4 Hz.

8. The method of claim 6, further comprising:
classifying, based on the one or more biological signals, the user's current sleep stage as a deep-sleep stage; and
providing the stimulation to the user in response to (1) the classification and (2) a determination that one or more sleep metrics of the user exceed one or more corresponding thresholds.

9. The method of claim 6, further comprising applying, during a wake sleep stage of the user, a cue stimulation to the user during a particular task performed by the user, wherein the acoustic or vibrational stimulation applied to the user comprises the cue stimulation.

10. The method of claim 1, further comprising:
skipping one or more windows in one or more segments of the obtained biological data; and
inferring one or more features for determining the sleep stage of the user in each skipped window based on at least one of (1) corresponding features in one or more windows prior to the skipped one or more windows or (2) corresponding features in one or more windows after the skipped one or more windows.

11. A system comprising:
one or more non-transitory computer readable storage media storing instructions, and one or more processors coupled to the one or more non-transitory computer readable storage media and operable to execute the instructions to:
obtain, by one or more sensors of a wearable device worn by a user, one or more biological signals of the user;
determine, based on the one or more biological signals, a current sleep stage of the user;
determine, based at least on the determined sleep stage of the user, whether to provide a stimulation to the user; and
in response to a determination to provide a stimulation to the user, then provide, by one or more electronic devices in the environment of the user, the stimulation to the user.

12. The system of claim 11, wherein the wearable device comprises a wrist-worn device.

13. The system of claim 12, wherein the one or more sensors comprise one or more of a PPG sensor, a heartrate sensor, an accelerometer, or a gyroscope.

14. The system of claim 13, wherein the one or more electronic devices comprise the wearable device.

15. The system of claim 11, further comprising one or more processors operable to the execute the instructions to:
determine, based on the one or more biological signal, that the user is currently in a deep-sleep sleep stage; and
in response to the determination, then provide an acoustic stimulation or a vibrational stimulation to the user.

16. The system of claim 15, wherein the acoustic stimulation comprises one or more acoustic pulses or one or more vibrational pulses having a pulse frequency in a range of 0.5 Hz to 4 Hz.

17. The system of claim 15, further comprising one or more processors operable to the execute the instructions to:
classify, based on the one or more biological signals, the user's current sleep stage as a deep-sleep stage; and
provide the stimulation to the user in response to (1) the classification and (2) a determination that one or more sleep metrics of the user exceed one or more corresponding thresholds.

18. The system of claim 15, further comprising one or more processors operable to the execute the instructions to apply, during a wake sleep stage of the user, a cue stimulation to the user during a particular task performed by the user, wherein the acoustic or vibrational stimulation applied to the user comprises the cue stimulation.

19. The system of claim 11, further comprising one or more processors operable to the execute the instructions to:
skip one or more windows in one or more segments of the obtained biological data; and
infer one or more features for determining the sleep stage of the user in each skipped window based on at least one of (1) corresponding features in one or more windows prior to the skipped one or more windows or (2) corresponding features in one or more windows after the skipped one or more windows.

20. One or more non-transitory computer-readable storage media comprising instructions that are operable when executed by one or more processors to:
obtain, by one or more sensors of a wearable device worn by a user, one or more biological signals of the user;
determine, based on the one or more biological signals, a current sleep stage of the user;
determine, based at least on the determined sleep stage of the user, whether to provide a stimulation to the user; and
in response to a determination to provide a stimulation to the user, then provide, by one or more electronic devices in the environment of the user, the stimulation to the user.
